(19) United States
(12) Patent Application Publication
(10) Pub. No.: US 2009/0274726 A1
(43) Pub. Date: Nov. 5, 2009

(54) SYNTHETIC GENE

(76) Inventors: Sara Jane Brett, Hertfordshire (GB); Michael Nail Burden, Hertfordshire (GB); Peter Franz Ertl, Hertfordshire (GB); Paul Andrew Hamblin, Hertfordshire (GB); John Philip Tite, Hertfordshire (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 12/439,880

(22) PCT Filed: Sep. 3, 2007
(86) PCT No.: PCT/EP07/59187

§ 371 Date: Mar. 4, 2009

(30) Foreign Application Priority Data
Sep. 4, 2006 (GB) 0617387.6

(51) Int. Cl. A61K 39/21; C12N 15/11; C12N 15/00; A61K 39/00; A61K 39/29; C12Q 1/68

(52) U.S. Cl. 424/208.1; 536/23.1; 435/320.1; 424/184.1; 424/228.1; 424/277.1; 435/6

(57) ABSTRACT

The present invention relates to synthetic genes, processes for designing said synthetic genes and their uses in gene therapy and improved DNA vaccination. The novel synthetic genes and processes are codon shuffled so that they have reduced homology relative to a naturally occurring gene encoding the same protein without altering the overall codon usage frequency of the gene. In particular the present invention relates to improved polynucleotides and methods for the treatment or prevention of disease comprising codon-shuffled GM-CSF nucleic acid sequences. Nucleic acid vaccines of the present invention may comprise a combination of a nucleotide sequence encoding codon-shuffled GM-CSF, a nucleotide encoding an antigen against which it is desired to raise an immune response and a toll-like receptor (TLR) agonist.

### BACKGROUND OF THE INVENTION

The present invention relates to synthetic genes, processes for designing said synthetic genes and their uses in gene therapy and improved DNA vaccination. The novel synthetic genes and processes are codon shuffled so that they have reduced homology relative to a naturally occurring gene encoding the same protein without altering the overall codon usage frequency of the gene. In particular the present invention relates to improved polynucleotides and methods for the treatment or prevention of disease comprising codon-shuffled GM-CSF nucleic acid sequences. Nucleic acid vaccines of the present invention may comprise a combination of a nucleotide sequence encoding codon-shuffled GM-CSF, a nucleotide encoding an antigen against which it is desired to raise an immune response and a toll-like receptor (TLR) agonist.

The present invention is in the field of medical therapy where a DNA polynucleotide is administered into a cell of a host. In the case of gene therapy and DNA vaccination, a gene encoding a protein is introduced into a host cell of a patient in order to obtain the therapeutic benefit, either the expression of the introduced DNA to obtain a functional product in the case of gene therapy or to express the product in order to trigger an immune response in the case of a vaccine. In either case, the DNA encoding the protein may have a high degree of homology to a gene which is already present in the genome of the host cell. There is a concern in the art that high degrees of homology of the introduced DNA and host cell gene could lead to safety issues associated with homologous recombination events which result in the introduced DNA incorporating into the host cell genome. The use of a wild type DNA sequence encoding a human protein as a component of a DNA vaccine for administration to a human cell therefore carries with it a very small risk of homologous recombination of plasmid DNA into the host genome.

Because of the concerns that introduced DNA may integrate into the host genome, and thereby raise the chance of carcinogenesis or chromosomal instability, the US Food and Drug Administration have issued draft guidance for the pharmaceutical industry indicating that experiments to evaluate in vivo biodistribution and potential integration of the administered plasmid are required during the application for product approval in order to address their concerns about this issue “Considerations for plasmid DNA vaccines for infectious disease indications February, 2005. (CBER)”.

The DNA code has 4 letters (A, T, C and G) and uses these to spell three letter “codons” which represent the amino acids the proteins encode in an organism's genes. The linear sequence of codons along the DNA molecule is translated into the linear sequence of amino acids in the protein (s) encoded by those genes. The code is highly degenerate, with 61 codons coding for the 20 natural amino acids and 3 codons representing “stop” signals. Thus, most amino acids are coded for by more than one codon-in fact several are coded for by four or more different codons.

Where more than one codon is available to code for a given amino acid, it has been observed that the codon usage patterns of organisms are highly non-random. Different species show a different bias in their codon selection and, furthermore, utilisation of codons may be markedly different in a single species between genes which are expressed at high and low levels. This bias is different in viruses, plants, bacteria and mammalian cells, and some species show a stronger bias away from a random codon selection than others. For example, humans and other mammals are less strongly biased than certain bacteria or viruses.

It is known that synthetic genes, which encode the same protein as a naturally occurring or wild type gene, may be designed by changing the codons that are used in the gene. These design techniques involve replacing those codons in a gene that are rarely used in mammalian genes with codons that are more frequently used for that amino acid in mammalian gene. This process, called codon optimisation, is used with the intent that the total level of protein produced by the host cell is greater when transfected with the codon-optimised gene in comparison with the level when transfected with the wild-type sequence. Several method have been published (Nakamura et. al., Nucleic Acids Research 1996, 24: 214-215; WO98/34640; WO97/11086).

Whether or not a gene has been codon optimised can be measured by an increase in the codon usage coefficient. The “codon usage coefficient” is a measure of how closely the codon pattern of a given polynucleotide sequence resembles that of a target species. Codon frequencies can be derived from literature sources for the highly expressed genes of many species (see e. g. Nakamura et al. Nucleic Acids Research 1996, 24; 214-215). The codon frequencies for each of the 61 codons (expressed as the number of occurrences per 1000 codons of the selected class of genes) are normalised for each of the twenty natural amino acids, so that the value for the most frequently used codon for each amino acid is set to 1 and the frequencies for the less common codons are scaled to lie between zero and 1. Thus each of the 61 codons is assigned a value of 1 or lower for the highly expressed genes of the target species. In order to calculate a codon usage coefficient for a specific polynucleotide, relative to the highly expressed genes of that species, the scaled value for each codon of the specific polynucleotide are noted and the geometric mean of all these values is taken (by dividing the sum of the natural logs of these values by the total number of codons and take the anti-log). The coefficient will have a value between zero and 1 and the higher the coefficient the more codons in the poly nucleotide are frequently used codons. Those polynucleotide sequences which have a codon usage coefficient close to 1 are thought to be more highly expressed in a mammalian cell than those sequences of a lower codon usage coefficient, such as for example 0.2.

The codon usage table for a homo sapiens is set out below:

Codon usage for human (highly expressed) genes 1/24/91 (human-high. cod) (WO2005025614)

| AmAcid | Codon | Number | /1000 | Fraction . . . |
|---|---|---|---|---|
| Gly | GGG | 905.00 | 18.76 | 0.24 |
| Gly | GGA | 525.00 | 10.88 | 0.14 |
| Gly | GGT | 441.00 | 9.14 | 0.12 |
| Gly | GGC | 1867.00 | 38.70 | 0.50 |
| Glu | GAG | 2420.00 | 50.16 | 0.75 |
| Glu | GAA | 792.00 | 16.42 | 0.25 |
| Asp | GAT | 592.00 | 12.27 | 0.25 |
| Asp | GAC | 1821.00 | 37.75 | 0.75 |
| Val | GTG | 1866.00 | 38.68 | 0.64 |
| Val | GTA | 134.00 | 2.78 | 0.05 |
| Val | GTT | 198.00 | 4.10 | 0.07 |
| Val | GTC | 728.00 | 15.09 | 0.25 |
| Ala | GCG | 652.00 | 13.51 | 0.17 |
| Ala | GCA | 488.00 | 10.12 | 0.13 |
| Ala | GCT | 654.00 | 13.56 | 0.17 |
| Ala | GCC | 2057.00 | 42.64 | 0.53 |
| Arg | AGG | 512.00 | 10.61 | 0.18 |
| Arg | AGA | 298.00 | 6.18 | 0.10 |
| Ser | AGT | 354.00 | 7.34 | 0.10 |
| Ser | AGC | 1171.00 | 24.27 | 0.34 |
| Lys | AAG | 2117.00 | 43.88 | 0.82 |
| Lys | AAA | 471.00 | 9.76 | 0.18 |
| Asn | AAT | 314.00 | 6.51 | 0.22 |
| Asn | AAC | 1120.00 | 23.22 | 0.78 |
| Met | ATG | 1077.00 | 22.32 | 1.00 |
| Ile | ATA | 88.00 | 1.82 | 0.05 |
| Ile | ATT | 315.00 | 6.53 | 0.18 |
| Ile | ATC | 1369.00 | 28.38 | 0.77 |
| Thr | ACG | 405.00 | 8.40 | 0.15 |
| Thr | ACA | 373.00 | 7.73 | 0.14 |
| Thr | ACT | 358.00 | 7.42 | 0.14 |
| Thr | ACC | 1502.00 | 31.13 | 0.57 |
| Trp | TGG | 652.00 | 13.51 | 1.00 |
| End | TGA | 109.00 | 2.26 | 0.55 |
| Cys | TGT | 325.00 | 6.74 | 0.32 |
| Cys | TGC | 706.00 | 14.63 | 0.68 |
| End | TAG | 42.00 | 0.87 | 0.21 |
| End | TAA | 46.00 | 0.95 | 0.23 |
| Tyr | TAT | 360.00 | 7.46 | 0.26 |
| Tyr | TAC | 1042.00 | 21.60 | 0.74 |
| Leu | TTG | 313.00 | 6.49 | 0.06 |
| Leu | TTA | 76.00 | 1.58 | 0.02 |
| Phe | TTT | 336.00 | 6.96 | 0.20 |
| Phe | TTC | 1377.00 | 28.54 | 0.80 |
| Ser | TCG | 325.00 | 6.74 | 0.09 |
| Ser | TCA | 165.00 | 3.42 | 0.05 |
| Ser | TCT | 450.00 | 9.33 | 0.13 |
| Ser | TCC | 958.00 | 19.86 | 0.28 |
| Arg | CGG | 611.00 | 12.67 | 0.21 |
| Arg | CGA | 183.00 | 3.79 | 0.06 |
| Arg | CGT | 210.00 | 4.35 | 0.07 |
| Arg | CGC | 1086.00 | 22.51 | 0.37 |
| Gln | CAG | 2020.00 | 41.87 | 0.88 |
| Gln | CAA | 283.00 | 5.87 | 0.12 |
| His | CAT | 234.00 | 4.85 | 0.21 |
| His | CAC | 870.00 | 18.03 | 0.79 |
| Leu | CTG | 2884.00 | 59.78 | 0.58 |
| Leu | CTA | 166.00 | 3.44 | 0.03 |
| Leu | CTT | 238.00 | 4.93 | 0.05 |
| Leu | CTC | 1276.00 | 26.45 | 0.26 |
| Pro | CCG | 482.00 | 9.99 | 0.17 |
| Pro | CCA | 456.00 | 9.45 | 0.16 |
| Pro | CCT | 568.00 | 11.77 | 0.19 |
| Pro | CCC | 1410.00 | 29.23 | 0.48 |

Traditional vaccination techniques which involve the introduction into an animal system of an antigen which can induce an immune response in the animal, and thereby protect the animal against infection, have been known for many years. Following the observation in the early 1990's that plasmid DNA could directly transfect animal cells in vivo, significant research efforts have been undertaken to develop vaccination techniques based upon the use of DNA plasmids to induce immune responses, by direct introduction into animals of DNA which encodes for antigenic peptides. Such techniques, which are referred to as “DNA immunisation” or “DNA vaccination” have now been used to elicit protective antibody (humoral) and cell-mediated (cellular) immune responses in a wide variety of pre-clinical models for viral, bacterial and parasitic diseases.

DNA vaccines usually consist of a bacterial plasmid vector into which is inserted a strong promoter, the gene of interest which encodes for an antigenic peptide against which it is desired to raise an immune response and a polyadenylation/transcriptional termination sequence. Alternatively the DNA vaccine may comprise a vector for effecting the expression of the gene of interest in a cell, such as for example a viral vector. The immunogen which the gene of interest encodes may be a fall protein or simply an antigenic peptide sequence relating to the pathogen, tumour or other agent which is intended to be protected against. The plasmid can be grown in bacteria, such as for example E. coli and then isolated and prepared in an appropriate medium, depending upon the intended route of administration, before being administered to the host.

Helpful background information in relation to DNA vaccination is provided in “Donnelly, J et al Annual Rev. Immunol. (1997) 15:617-648; Ertl P. and Thomsen L., Technical issues in construction of nucleic acid vaccines Methods. 2003 November; 31(3):199-206; the disclosures of which are included herein in their entirety by way of reference.

Despite the numerous successes of DNA vaccination relative to traditional vaccination therapies, there is nonetheless a desire to develop adjuvant compounds which will serve to increase the immune response induced by the protein which is encoded by the plasmid DNA administered to an animal.

Granulocyte-macrophage colony stimulating factor (GM-CSF) is a cytokine capable of inducing differentiation, proliferation and activation of a range of cells with immunological function. GM-CSF induces proliferation of dendritic cells from bone marrow precursors to reach an immature dendritic cell state, i.e. the cells express low levels of co-stimulatory markers and high levels of receptors for antigen uptake.

The use of GM-CSF in vaccines is already known in the art (U.S. Pat. No. 5,679,356) and additionally the sequence of GM-CSF has been codon optimised (WO 04/004742) in order to yield a more highly expressed protein. This optimised nucleotide sequence therefore has a higher codon usage coefficient for expression in a mammalian gene. The present invention provides a synthetic gene encoding human GM-CSF with reduced homology to the constitutive human GM-CSF gene, thereby reducing the risk of the synthetic gene recombining with the human genome when the synthetic gene is inserted into human cell during gene therapy or DNA vaccination.

Toll-like receptors (TLRs) are type I transmembrane receptors, evolutionarily conserved between insects and humans. Ten TLRs have so far been established (TLRs 1-10) (Sabroe et al, JI 2003 p 1630-5). Members of the TLR family have similar extracellular and intracellular domains; their extracellular domains have been shown to have leucine-rich repeating sequences, and their intracellular domains are similar to the intracellular region of the interleukin-1 receptor (IL-1R). TLR cells are expressed differentially among immune cells and other cells (including vascular epithelial cells, adipocytes, cardiac myocytes and intestinal epithelial cells). The intracellular domain of the TLRs can interact with the adaptor protein Myd88, which also posses the IL-1R domain in its cytoplasmic region, leading to NP-KB activation of cytokines; this Myd88 pathway is one way by which cytokine release is effected by TLR activation. The main expression of TLRs is in cell types such as antigen presenting cells (e.g. dendritic cells, macrophages etc).

Activation of dendritic cells by stimulation through the TLRs leads to maturation of dendritic cells, and production of inflammatory cytokines such as IL-12. Research carried out so far has found that TLRs recognise different types of agonists, although some agonists are common to several TLRs. TLR agonists are predominantly derived from bacteria or viruses, and include molecules such as flagellin or bacterial lipopolysaccharide (LPS).

The imidazoquinoline compounds imiquimod and resiquimod are small anti-viral compounds. Imiquimod has been used for the local treatment of genital warts caused by human papilloma virus; resiquimod has also been tested for use in treatment of genital warts. Imiquimod and resiquimod are believed to act through the TLR-7 and/or TLR-8 signalling pathways and activation of the Myd88 activation pathway.

There remains a need in the art to provide novel DNA compositions for administration into a host cell, where the genome of the host cell comprises a gene that encodes the same protein as the DNA composition and that have a reduced risk associated with homologous recombination of the composition into the genome of a host cell.

### SUMMARY OF THE INVENTION

The present invention provides novel non-naturally occurring genes, and processes to design the synthetic genes, that have a greatly reduced homology in comparison to a gene present in the mammalian genome. In the case of gene therapy or DNA vaccination of a human, the novel synthetic gene has a greatly reduced homology in comparison to a gene present in the mammalian genome that encodes the same protein.

In general, the techniques of the present invention allow the production of non naturally occurring genes having a lower degree of homology than are obtained using other codon modification techniques in the art.

According to one embodiment of the present invention there is provided a non-naturally occurring DNA sequence that encodes a protein wherein the non-naturally occurring gene comprises a DNA sequence having less than 80% homology to a wild-type cDNA sequence or coding sequence encoding the same protein, characterized in that the codon usage coefficient for the non-naturally occurring gene is not greater than the wild type cDNA sequence.

Within the context of all aspects of this invention “non-naturally occurring sequence” is an artificial sequence that has been designed for the purpose of this invention, and is not found in toto within the genome of a mammal.

In one embodiment the non-naturally occurring DNA sequence encodes a human protein.

In another embodiment the non naturally occurring DNA sequences encodes human GM-CSF.

In the context of the present invention the non naturally occurring DNA sequence encodes a protein which is biologically active when expressed by a transfected host cell. The biological activity may be either a) the protein has the activity of the protein as if it were produced by the wild-type gene in the genome of the host cell, or b) it is active in that it is capable of stimulating an immune response that is specific for the protein produced by the wild type gene in the genome of the host cell.

It will be appreciated that the non-naturally occurring sequence may encode the biologically active, or mature, form of the protein, whereas the wild type gene present in the genome of the host cell may encode an inactive protein which is activated on a post translational event. The skilled man will also appreciate that the host cell gene may comprise coding and non-coding regions (exons and introns respectively) whereas the mRNA produced during expression of the gene will only contain complementary RNA sequences to the coding regions of the gene. Complementary DNA (or cDNA) sequences which may be produced or deduced from the mRNA corresponds, therefore to the coding regions of the host cell gene.

In one embodiment the non-naturally occurring sequences of the present invention encode either the mature form of the protein, or it encodes the inactive pre-processed form of the protein. The non naturally occurring sequences of the present invention do not contain the introns that may appear in the host cell gene for the same protein, and as such the skilled man will appreciate that the degree of homology of the non naturally occurring sequence should be calculated relative to the coding portion of the host cell, or wild type gene which corresponds to the cDNA sequence that encodes the same protein as the non naturally occurring sequence.

In one embodiment of the present invention there is provided a non-naturally occurring gene that encodes a protein wherein the non-naturally occurring gene comprises a DNA sequence having less than 80% homology to a cDNA sequence encoding the same wild type protein, characterized in that the codon usage coefficient for the non-naturally occurring gene is the same as the wild type gene.

In one embodiment the level of production of the protein by a host cell which is transfected with said non-naturally occurring gene, in a form which allows expression thereof, is no greater than the level of protein produced by a host cell when it is transfected with the wild type gene.

In one embodiment the non-naturally occurring gene encodes a polypeptide which is identical to the mature form of a polypeptide which is encoded by a gene present in the human genome.

In one embodiment the non-naturally occurring gene encodes a polypeptide which is identical to the precursor form of a polypeptide which is encoded by a gene present in the human genome. By precursor is meant the polypeptide sequence including its signal or pro sequence.

In one embodiment the non naturally occurring gene encodes codon-shuffled Granulocyte Macrophage Colony Stimulating factor (GM-CSF).

Within the context of all aspects of this invention by GM-CSF is meant the entire molecule of GM-CSF either in mature form or precursor form with its signal sequence or any fragment thereof capable of inducing proliferation of bone marrow precursor cells to reach an immature dendritic cell state. The wild type DNA sequence for human GM-CSF is found in the Genbank database (accession number M11220-Ref. Lee, F. et al PNAS 82 (13) 4360-4364 (1985)).

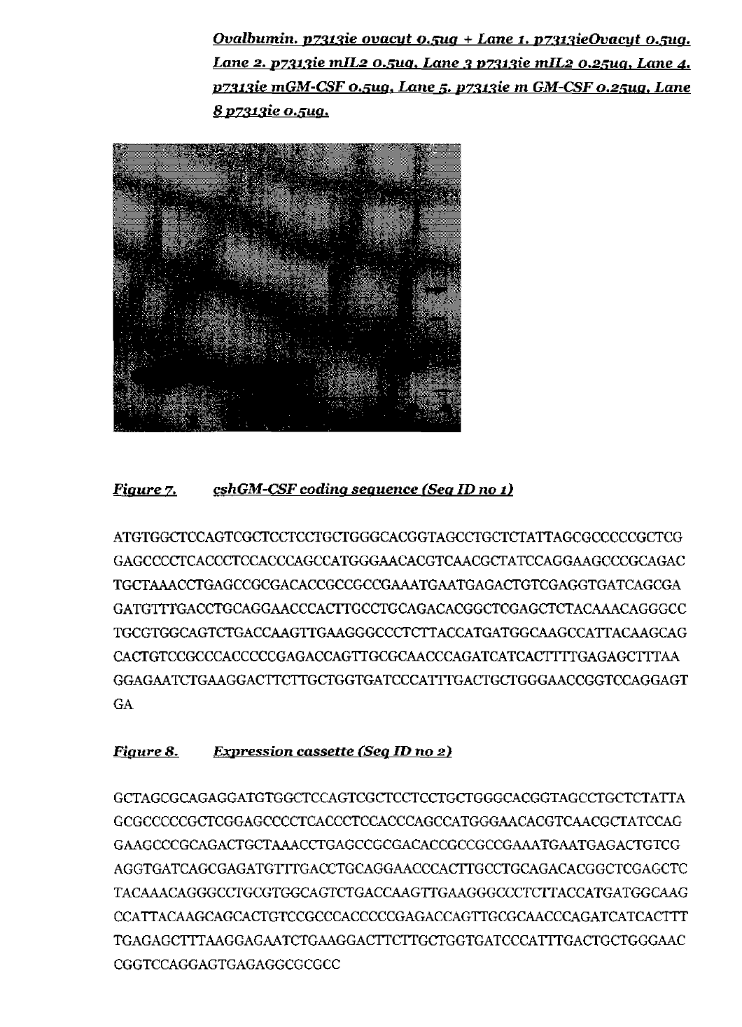

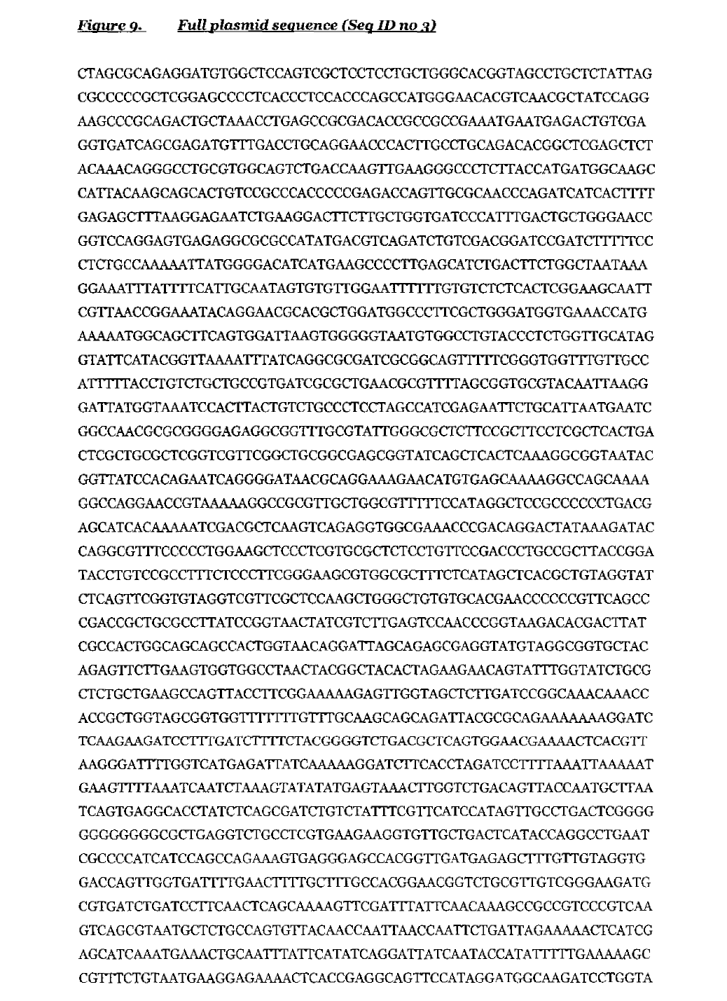

In one embodiment the sequence of the non naturally occurring GM-CSF gene is that of SEQ ID NO. 1. (FIG. 7)

In one embodiment the non-naturally occurring gene is comprised in an expression cassette, comprising the non naturally occurring gene and the regulatory control sequences.

In another embodiment of the present invention the expression cassette containing the non-naturally occurring gene may be comprised in a plasmid.

The nucleotide sequences of the present invention, for example the nucleotide sequence encoding GM-CSF, may be provided within the context of a plasmid comprising regulatory control sequences. For example, the nucleotide sequence may be within the context of vaccine vector p7313 (details included in WO 02/08435) under the regulatory control of human cytomegalovirus (Cm major immediate early (IE) promoter.

The present invention also provides expression vectors that comprise the non naturally occurring genes of the present invention. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for protein expression. Other suitable vectors would be apparent to persons skilled in the art. By way of further example in this regard we refer to Sambrook et al. Molecular Cloning: a Laboratory Manual. 2nd Edition. CSH Laboratory Press. (1989).

A vector comprising the non naturally occurring polynucleotide, for use in the invention, may be operably linked to a control sequence which is capable of providing the expression of the coding sequence by the host cell, i. e. the vector is an expression vector. The term “operably linked” refers to a juxtaposition wherein the components described are in a relationship permitting them to function in their intended manner. A regulatory sequence, such as a promoter, “operably linked” to a coding sequence is positioned in such a way that expression of the coding sequence is achieved under conditions compatible with the regulatory sequence.

The vectors may be, for example, plasmids, artificial chromosomes (e. g. BAC, PAC, YAC), virus or phage vectors provided with an origin of replication, optionally a promoter for the expression of the polynucleotide and optionally a regulator of the promoter. The vectors may contain one or more selectable marker genes, for example an ampicillin or kanamycin resistance gene in the case of a bacterial plasmid or a resistance gene for a fungal vector. Vectors may be used in vitro, for example for the production of DNA or RNA or used to transfect or transform a host cell, for example, a mammalian host cell for the production of protein encoded by the vector. The vectors may also be adapted to be used in vivo, for example in a method of DNA vaccination or of gene therapy.

Promoters and other expression regulation signals may be selected to be compatible with the host cell for which expression is designed. For example, mammalian promoters include the metallothionein promoter, which can be induced in response to heavy metals such as cadmium, and the p-actin promoter. Viral promoters such as the SV40 large T antigen promoter, human cytomegalovirus (CMV) immediate early (IE) promoter, rous sarcoma virus LTR promoter, adenovirus promoter, or a HPV promoter, particularly the HPV upstream regulatory region (URR) may also be used. All these promoters are well described and readily available in the art.

One promoter element is the CMV immediate early promoter devoid of intron A, but including exon 1 (WO02/36792). Accordingly there is provided a vector comprising a polynucleotide of the invention under the control of HCMV IE early promoter.

Examples of suitable viral vectors include herpes simplex viral vectors, vaccinia or alpha-virus vectors and retroviruses, including lentiviruses, adenoviruses and adeno-associated viruses. Gene transfer techniques using these viruses are known to those skilled in the art. Retrovirus vectors for example may be used to stably integrate the polynucleotide of the invention into the host genome, although such recombination is not preferred.

Replication-defective adenovirus vectors by contrast remain episomal and therefore allow transient expression. Vectors capable of driving expression in insect cells (for example baculovirus vectors), in human cells or in bacteria may be employed in order to produce quantities of the HIV protein encoded by the polynucleotides of the present invention, for example for use as subunit vaccines or in immunoassays. The polynucleotides of the invention have particular utility in viral vaccines as previous attempts to generate full length vaccinia constructs have been unsuccessful.

In one embodiment of the present invention, viral vectors may be used which comprise an adenoviral nucleic acid sequence selected from C1, Pan 5, Pan 6, Pan 7, C68 (Pan 9), SV1, SV25 and SV39, as described in published PCT application WO 03/046124, the entirety of which earlier publication is incorporated herein by reference.

Bacterial vectors, such as attenuated Salmonella or Listeria may alternatively be used.

In one embodiment of the present invention there is provided a vaccine composition comprising:
a) an immunogen component
b) adjuvant components

wherein either a) or b) or both can be codon shuffled genes.

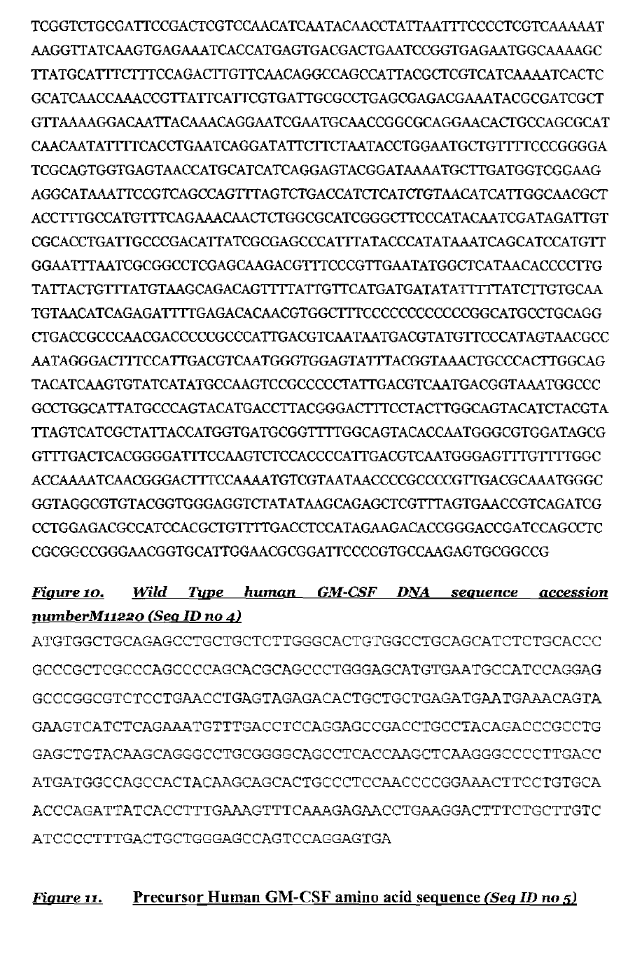

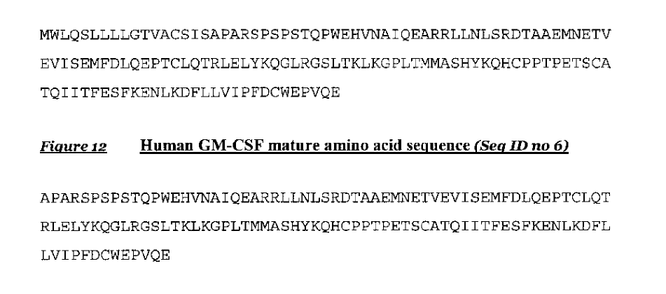

In one embodiment, where the non naturally occurring gene sequence is for use in human vaccines, the wild-type human GM-CSF sequence is shown in SEQ ID 4 (see FIG. 10). The present invention further provides a vaccine composition or compositions comprising a plasmid containing a nucleotide sequence encoding an antigenic peptide or protein and a plasmid containing a polynucleotide sequence encoding codon-shuffled GM-CSF. The invention further provides a method of vaccinating a mammalian subject which comprises administering thereto an effective amount of such a vaccine or vaccine composition. Expression vectors for use in DNA vaccines, vaccine compositions and immunotherapeutics may be plasmid vectors.

In another embodiment of the present invention the polynucleotide may encode a protein against which it is desired to induce an immune response whereby the polynucleotide encodes for antigens selected from HCV, HIV, HPV or a tumor associated antigen.

The nucleotide sequences of the immunogen component referred to in this application, encoding antigen or immunogen to be expressed within a mammalian system, in order to induce an antigenic response, may encode for an entire protein, or merely a shorter peptide sequence which is capable of initiating an antigenic response. Throughout this specification and the appended claims, the phrase “antigenic peptide” or “immunogen” is intended to encompass all peptide or protein sequences which are capable of inducing an immune response within the animal concerned. In one embodiment, however, the nucleotide sequence will encode for a full protein which is associated with the disease state, as the expression of full proteins within the animal system are more likely to mimic natural antigen presentation, and thereby evoke a full immune response. Some non-limiting examples of known antigenic peptides in relation to specific disease states include the following:

In one embodiment the antigens used in the present invention may be capable of eliciting an immune response against a human pathogen, such as viral, bacterial or parasitic antigens.

It is possible for the vaccination methods and compositions according to the present application to be adapted for protection or treatment of mammals against a variety of disease states such as, for example, viral, bacterial or parasitic infections, cancer, allergies and autoimmune disorders. Some specific examples of disorders or disease states which can be protected against or treated by using the methods or compositions according to the present invention, are as follows: Viral Infections Hepatitis viruses A, B, C, D & E, HIV, herpes viruses 1,2 6 & 7,-cytomegalovirus, varicella zoster, papilloma virus, Epstein Barr virus, influenza viruses, para-influenza viruses, adenoviruses, coxsakie viruses, picorna viruses, rotaviruses, respiratory syncytial viruses, pox viruses, rhinoviruses, rubella virus, papovirus, mumps virus, measles virus.

Bacterial Infections Mycobacteria causing TB and leprosy, pneumocci, aerobic gram negative bacilli, mycoplasma, staphyloccocal infections, streptococcal infections, salmonellae, chlamydiae.

Parasitic Malaria, leishmaniasis, trypanosomiasis, toxoplasmosis, schistosomiasis, filariasis, Cancer Breast cancer, colon cancer, rectal cancer, cancer of the head and neck, renal cancer, malignant melanoma, laryngeal cancer, ovarian cancer, cervical cancer, prostate cancer.

Allergies Rhinitis due to house dust mite, pollen and other environmental allergens Autoimmune disease Systemic lupus erythematosis In one embodiment, the methods or compositions of the present invention are used to protect against or treat the viral disorders Hepatitis B, Hepatitis C, Human papilloma virus, Human immunodeficiency virus, or Herpes simplex virus; the bacterial disease TB; cancers of the breast, colon, ovary, cervix, and prostate; or the autoimmune diseases of asthma rheumatoid arthritis and Alzheimer's. It is to be recognised that these specific disease states have been referred to by way of example only, and are not intended to be limiting upon the scope of the present invention.

In a further embodiment of the present invention, the codon shuffled GM-CSF encoded by a nucleotide sequence, and the nucleotide sequences encoding the immunogen component are comprised or consist within separate polynucleotide molecules, for concomitant or sequential administration. In an embodiment of the invention where immunogen and codon shuffled GM-CSF components are comprised or consist within separate polynucleotide molecules, the polynucleotide molecules may each be present within separate plasmids for concomitant or sequential delivery. In one embodiment, concomitant delivery may be used.

By concomitant administration is meant substantially simultaneous administration; that is, components are administered at the same time, or if not, at least within a few minutes of each other. Alternatively, components are administered within one, two, three, four, five or ten minutes of each other. In one treatment protocol, adjuvant component is administered substantially simultaneously to administration of the nucleotide sequence encoding immunogen and the codon shuffled GM-CSF. Obviously, this protocol can be varied as necessary. In one embodiment of the present invention, the adjuvant component is an imidazoquinoline or derivative thereof, and is provided in a separate composition from immunogen component and codon shuffled GM-CSF for concomitant or sequential administration. In one embodiment, the imidazoquinoline compound, or derivative thereof is administered sequentially, that is after the administration of the immunogen component and codon shuffled GM-CSF in a separate composition. In a further embodiment, the imidazoquinoline compound, or derivative thereof, is given 2, 4, 6, 8, 12 or 24 hours after administration of immunogen component and codon shuffled GM-CSF. In one embodiment, the imidazoquinoline compound or derivative thereof is given at or about 24 hours after administration of immunogen component and codon shuffled GM-CSF. In a further embodiment, where the imidazoquinoline compound, or derivative thereof is for topical administration, in a cream formulation, the cream is applied 24 hours after administration of the immunogen component and codon shuffled GM-CSF.

In an alternative embodiment of the present invention, where the imidazoquinoline compound, or derivative thereof is provided in a soluble formulation for administration, for example but not limited to sub-cutaneous administration, the imidazoquinoline compound, or derivative thereof may be administered between 6 and 24 hours after administration of the immunogen component and codon-shuffled GM-CSF, or may be administered the next working day after administration of the immunogen component and codon-shuffled GM-CSF. Immunogen component and codon-shuffled GM-CSF may be packaged onto a gold bead and administered into the skin of a patient using particle mediated drug delivery, for example using a “gene gun” as described in, for example, EP0500799.

Alternatively, the present invention provides a pharmaceutical composition or compositions comprising an immunogenic composition or compositions as described herein, and pharmaceutically acceptable excipients, diluents or carriers

The invention further provides a pharmaceutical composition or compositions comprising adjuvant component according to the present invention; an immunogen component comprising a nucleotide sequence encoding an antigenic peptide or protein; and one or more pharmaceutically acceptable excipients, diluents or carriers.

In a further embodiment of the present invention the method of inducing an immune response in a mammal, comprising administering to said mammal the codon shuffled polynucleotides of the invention, further comprises the administration of a TLR-agonist.

By “TLR agonist” it is meant a component which is capable of causing a signalling response through a TLR signalling pathway, either as a direct ligand or indirectly through generation of endogenous or exogenous ligand (Sabroe et al, JI 2003 p 1630-5).

In an embodiment of the present invention, the TLR agonist is capable of causing a signalling response through TLR-7. In one embodiment of the present invention, the TLR agonist is an imidazoquinoline compound, or derivative thereof. In a further embodiment, the imidazoquinoline or derivative thereof is a compound defined by any one of formulae I-VI, as defined herein. In a further embodiment, the imidazoquinoline or derivative thereof is a compound defined by formula VI. In one embodiment, the imidazoquinoline or derivative thereof is a compound of formula VI selected from the group consisting of 1-(2-methylpropyl)-1H-imidazo [4,5-c] quinolin-4-amine; 1-(2-hydroxy-2-methylpropyl)-2-methyl-1H-imidazo [4,5-c] quinolin-4-amine ;1-(2, hydroxy-2-methylpropyl)-1H-imidazo [4,5-c] quinolin-4-amine; 1-(2-hydroxy-2-methylpropyl)-2-ethoxymethyl-1-H-imidazo [4,5-c] quinolin-4-amine In a further embodiment the imidazoquinoline or derivative thereof is imiquimod or resiquimod. The imidazoquinoline or derivative thereof may be imiquimod.

The present invention further provides a kit comprising a pharmaceutical composition comprising an immunogen component and codon-shuffled GM-CSF component, and a pharmaceutically acceptable excipient, diluent or carrier; and a further pharmaceutical composition comprising a TLR agonist, or a nucleotide encoding a TLR agonist and a “carrier”. In one embodiment, at least one carrier is a gold bead and at least one pharmaceutical composition is amenable to delivery by particle mediated drug delivery.

The present invention further provides a method of raising an immune response in a mammal against a disease state, comprising administering to the mammal within an appropriate vector, a nucleotide sequence encoding an antigenic peptide associated with the disease state; additionally administering to the mammal within an appropriate vector, a nucleotide sequence encoding codon shuffled GM-CSF; and further administering to the mammal an imidazoquinoline or derivative thereof to raise the immune response.

The present invention further provides a method of increasing the immune response of a mammal to an immunogen, comprising the step of administering to the mammal within an appropriate vector, a nucleotide sequence encoding the immunogen in an amount effective to stimulate an immune response and a nucleotide sequence encoding codon shuffled GM-CSF; and further administering to the mammal an imidazoquinoline or derivative thereof in an amount effective to increase the immune response.

In one embodiment of the invention is provided a method of creating a synthetic gene that has less than 80% homology at a DNA level to a cDNA sequence of a wild-type gene wherein the synthetic gene and wild-type EDNA gene encode the same polypeptide comprising:
a) identifying the codons for each amino acid in the wild-type cDNA sequence; and
b) if any amino acid residue is encoded by the wild-type cDNA sequence in at least two locations then translocating at least 80% of the codons from its first position where it is found in the wild-type cDNA sequence to a second position not found in the wild-type cDNA sequence.

In this embodiment at least 90% of the codons have been translocated.

In one embodiment of the invention is provided a method of creating a synthetic gene that has less than 80% homology at a DNA level to a cDNA sequence of a wild-type gene wherein the synthetic gene and wild-type cDNA encode the same polypeptide comprising:
a) identifying the codons for each amino acid in the wild-type cDNA sequence; and
b) translocating the codons so that each codon is found in the synthetic gene in the same proportions as the wild-type cDNA sequence;

wherein the level of protein produced by the cell is not greater than that of the cell when transfected with the wild type cDNA sequence.

In one embodiment of the invention is provided a method of creating a synthetic gene that has less than 80% homology at a DNA level to a cDNA sequence of a wild-type gene wherein the synthetic gene and wild-type gene encode the same polypeptide comprising:
a) identifying the codons for each amino acid in the wild-type cDNA sequence; and
b) translocating a sufficient number of codons from their first position in the wild-type EDNA sequence to a second position not found in the wild-type cDNA sequence;
Wherein the codon usage coefficient of the synthetic genes is not higher than that of the wild-type cDNA sequence.

In one embodiment of the present invention is provided a method of treating a patient comprising the administration of a safe and effective amount of an immunogenic, vaccine or pharmaceutical composition according to the invention.

In one embodiment of the present invention is provided use of the plasmid of the invention and an imidazoquinoline or derivative thereof in the manufacture of a medicament for enhancing immune responses initiated by an antigenic peptide or protein, the antigenic peptide or protein being expressed as a result of administration to a mammal of a nucleotide sequence encoding for the peptide.

The present invention further provides the use of an immunogen and codon-shuffled GM-CSF components in the manufacture of a medicament for the enhancement of an immune response to an antigen encoded by a nucleotide sequence.

As used herein the term immunogenic composition refers to a combination of a nucleotide sequence encoding GM-CSF; and an immunogen component comprising a nucleotide sequence encoding an antigenic peptide or protein in which the components act in functional co-operation to enhance the immune responses in a mammal to the immunogen component.

If the immunogen component comprises a vector which comprises the nucleotide sequence encoding an antigenic peptide can be administered in a variety of manners. It is possible for the vector to be administered in a naked form (that is as naked nucleotide sequence not in association with liposomal formulations, with viral vectors or transfection facilitating proteins) suspended in an appropriate medium, for example a buffered saline solution such as PBS and then injected intramuscularly, subcutaneously, intraperitonally or intravenously, although some earlier data suggests that intramuscular or subcutaneous injection may be used (Brohm et al Vaccine 16 No. 9/10 pp 949-954 (1998), the disclosure of which is included herein in its entirety by way of reference). It is additionally possible for the vectors to be encapsulated by, for example, liposomes or within polylactide co-glycolide (PLG) particles (25) for administration via the oral, nasal or pulmonary routes in addition to the routes detailed above.

It is also possible, according to one embodiment of the invention, for intradermal administration of the immunogen component, for example via use of gene-gun (particularly particle bombardment) administration techniques. Such techniques may involve coating of the immunogen component on to gold beads which are then administered under high pressure into the epidermis, such as, for example, as described in Haynes et al J. Biotechnology 44: 37-42 (1996).

In one illustrative example, gas driven particle acceleration can be achieved with devices such as those manufactured by Powderject Pharmaceuticals PLC (Oxford, UK) and Powderject Vaccines Inc. (Madison, Wis.), some examples of which are described in U.S. Pat. Nos. 5,846,796; 6,010,478; 5,865,796; 5,584,807; and EP Patent No. 0500 799.

This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles may be gold beads of a 0.4-4.0, um, or 0.6-2.0 um diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412.

The nucleic acid vaccine may also be delivered by means of micro needles, which may be coated with a composition of the invention or delivered via the micro-needle from a reservoir. The vectors which comprise the nucleotide sequences encoding antigenic peptides are administered in such amount as will be prophylactically or therapeutically effective. The quantity to be administered, is generally in the range of one picogram to 1 milligram, or 1 picogram to 10 micrograms for particle-mediated delivery, and 10 micrograms to 1 milligram for other routes of nucleotide per dose. The exact quantity may vary considerably depending on the species and weight of the mammal being immunised, the route of administration, the potency and dose of the adjuvant components, the nature of the disease state being treated or protected against, the capacity of the subject's immune system to produce an immune response and the degree of protection or therapeutic efficacy desired. Based upon these variables, a medical or veterinary practitioner will readily be able to determine the appropriate dosage level.

It is possible for the immunogen component comprising the nucleotide sequence encoding the antigenic peptide, and the adjuvant components to be administered on a once off basis or to be administered repeatedly, for example, between 1 and 7 times, or between 1 and 4 times, at intervals between about 4 weeks and about 18 months. Once again, however, this treatment regime will be significantly varied depending upon the size of the patient, the disease which is being treated/protected against, the amount of nucleotide sequence administered, the route of administration, and other factors which would be apparent to a skilled medical practitioner. The patient may receive one or more other anti cancer drugs as part of their overall treatment regime.

Once again, depending upon the type of variables listed above, the dose of administration of the TLR agonist will also vary, but may, for example, range between about 0.1 mg per kg to about 100 mg per kg, where “per kg” refers to the body weight of the mammal concerned. This administration of the TLR agonist amine derivative may be repeated with each subsequent or booster administration of the nucleotide sequence. The administration dose may be between about 0.5 mg per kg to about 5 mg per kg, or about 1 mg/kg or 1 mg/kg. Where the TLR agonist is resiquimod or imiquimod, the dose may be in mg/kg. Where the TLR agonist is imiquimod, Aldara cream (5% imiquimod; 3M) may be used, and applied topically at or near the site of administration. In one embodiment of the invention, one 12.5 mg packet (3M) of 5% Aldara cream may be used, alternatively more than one packet of Aldara cream may be used. In a further embodiment of the invention, a fraction of a packet may be used: for example at or about 20%, 25%, 33% or 50% of a packet may be used at or near each site.

While it is possible for the TLR agonist adjuvant component to comprise an imidazoquinoline molecule or derivative thereof to be administered in the raw chemical state, administration may be in the form of a pharmaceutical formulation. That is, the TLR agonist adjuvant component may comprise the imidazoquinoline molecule or derivative thereof combined with one or more pharmaceutically or veterinarily acceptable carriers, and optionally other therapeutic ingredients. The carrier (s) must be “acceptable” in the sense of being compatible with other ingredients within the formulation, and not deleterious to the recipient thereof. The nature of the formulations will naturally vary according to the intended administration route, and may be prepared by methods well known in the pharmaceutical art. All methods of preparing formulations include the step of bringing into association an imidazoquinoline molecule or derivative thereof with an appropriate carrier or carriers. Carriers include a cream formulation, or alternatively PBS or water. In general, the formulations are prepared by uniformly and intimately bringing into association the derivative with liquid carriers or finely divided solid carriers, or both, and then, if necessary, shaping the product into the desired formulation. Formulations of the present invention suitable for oral administration may be presented as discrete units such as capsules, cachets or tablets each containing a pre-determined amount of the active ingredient; as a powder or granules; as a solution or a suspension in an aqueous liquid or a non-aqueous liquid; or as an oil-in-water liquid emulsion or a water-in-oil emulsion.

Administration of the adjuvant may take place between about 14 days prior to and about 14 days post administration of the nucleotide sequence, or between about 1 day prior to and about 3 days post administration of the nucleotide sequence. Nucleotide sequence encoding GM-CSF may be administered concomitantly with the administration of the nucleotide sequence encoding immunogen, and the component which is a TLR agonist provided sequentially. The component which is a TLR agonist may be given about or exactly 7, 6, 5, 4, 3, 2, or 1 day (s) or about or exactly 24.22, 20.18, 16.14, 12.10, 9.8, 7.6, 5.4, 3.2, or one hour (s) before the antigen component. The component which is a TLR agonist may be given about or exactly 7, 6, 5, 4, 3, 2 or 1 day (s) or about or exactly 24, 22, 20, 18, 16, 14, 12, 10, 9, 8, 7, 6, 5, 4, 3, 2, or one hour (s) after the antigen component.

The component which is a TLR agonist may be given at or about 24 hours after the remaining components. An advantage of giving the TLR agonist component after administration of the immunogen and codon-shuffled GM-CSF components and is that delivery of these components may lead to induction of IFNy in the locality of delivery; this may lead to upregulation of TLRs, such as up-regulation of TLRs 7 and/or 8, leading to increased responsiveness to the TLR agonist.

In one embodiment of the present invention, the immunogen and codon shuffled GM-CSF components are in a formulation suitable for simultaneous administration by gene gun delivery, and adjuvant component is provided in a separate cream formulation, for sequential topical administration.

Suitable techniques for introducing the naked polynucleotide or vector into a patient also include topical application with an appropriate vehicle. The nucleic acid may be administered topically to the skin or to mucosal surfaces for example by intranasal, oral, intravaginal or intrarectal administration. The naked polynucleotide or vector may be present together with a pharmaceutically acceptable excipient, such as phosphate buffered saline (PBS). DNA uptake may be further facilitated by use of facilitating agents such as bupivacaine, either separately or included in the DNA formulation. Other methods of administering the nucleic acid directly to a recipient include ultrasound, electrical stimulation, electroporation and microseeding which is described in U.S. Pat. No. 5,697,901.

Uptake of nucleic acid constructs may be enhanced by several known transfection techniques, for example those including the use of transfection agents. Examples of these agents includes cationic agents, for example, calcium phosphate and DEAE—Dextran and lipofectants, for example, lipofectam and transfectam. The dosage of the nucleic acid to be administered can be altered.

A nucleic acid sequence of the present invention may also be administered by means of transformed cells. Such cells include cells harvested from a subject. The naked polynucleotide or vector of the present invention can be introduced into such cells in vitro and the transformed cells can later be returned to the subject. The polynucleotide of the invention may integrate into nucleic acid already present in a cell by homologous recombination events. A transformed cell may, if desired, be grown up in vitro and one or more of the resultant cells may be used in the present invention. Cells can be provided at an appropriate site in a patient by known surgical or microsurgical techniques (e. g. grafting, micro-injection, etc.) The present inventors have demonstrated that the combination of TLR agonist with GM-CSF, when used as adjuvants in DNA vaccination, is capable of increasing cell-mediated immunology responses, in particular after a prime injection. The term adjuvant or adjuvant component as used herein is intended to convey that the derivatives or component comprising the derivatives act to enhance and/or alter the body's response to an immunogen in a desired fashion. So, for example, an adjuvant may be used to shift an immune response to a predominately Th1 response, or to increase both types of responses.

Throughout this specification and the appended claims, unless the context requires otherwise, the words “comprise” and “include” or variations such as “comprising”, “comprises”, “including”, “includes”, etc. , are to be construed inclusively, that is, use of these words will imply the possible inclusion of integers or elements not specifically recited. Additionally, the terms ‘comprising’, ‘comprise’ and ‘comprises’ herein is intended to be optionally substitutable by the terms ‘consisting of’, ‘consist of’ and ‘consists of’, respectively, in every instance.

The invention will now be described further, with reference to the following non-limiting examples:

### EXAMPLES

### Example 1

### Design of a Gene Encoding Codon Shuffled Human GM-CSF

The human GM-CSF DNA sequence was broken down into its constituent codons and these were pooled by their corresponding amino acid. For example the gene contains nine alanine residues represented by the codons GCA (x2), GCC (x5) and GCT (x2). The codons were manually reassigned in a different order to create a new DNA sequence which will translate the same amino acid sequence but would have reduced homology to the original wild-type sequence. Codons were assigned from the pools so that wherever possible a different codon was used relative to the corresponding co don in the wild type gene sequence. Once the initial reassignment of codons was complete, an alignment comparison with the wild type sequence was made and where necessary individual codon swaps made manually in order to A) ensure there were no stretches of identity greater than 20 base pairs and B) ensure that no clustering of the rare codons had occurred which might reduce the efficiency of translation.

This process resulted in a codon shuffled sequence for GM-CSF which maintains the overall codon usage as the wild type sequence but has only 76.8% homology to the wild type human GM-CSF DNA sequence, thus reducing the chance of homologous recombination and integration into the human host cells.

### Example 2

### Expression of Human GM-CSF from pMNB003 and pMNB004

In order to confirm expression of GM-CSF from the codon shuffled and wild-type GM-CSF plasmids, a number of DNA batches were made for each of the codon shuffled and wild-type human GSM-CSF plasmids and used to transiently transfect HEK293 cells. After 48 hours the supernatants were harvested and a human GM-CSF ELISA performed to compare the expression of the two constructs. The human GM-CSF ELISA was supplied as a kit of matched antibody pair, human GM-CSF standard and enzyme conjugate by R&D Systems (catalogue number DY215). FIG. 1 shows the standard curve for an ELISA using the standard components in the kit.

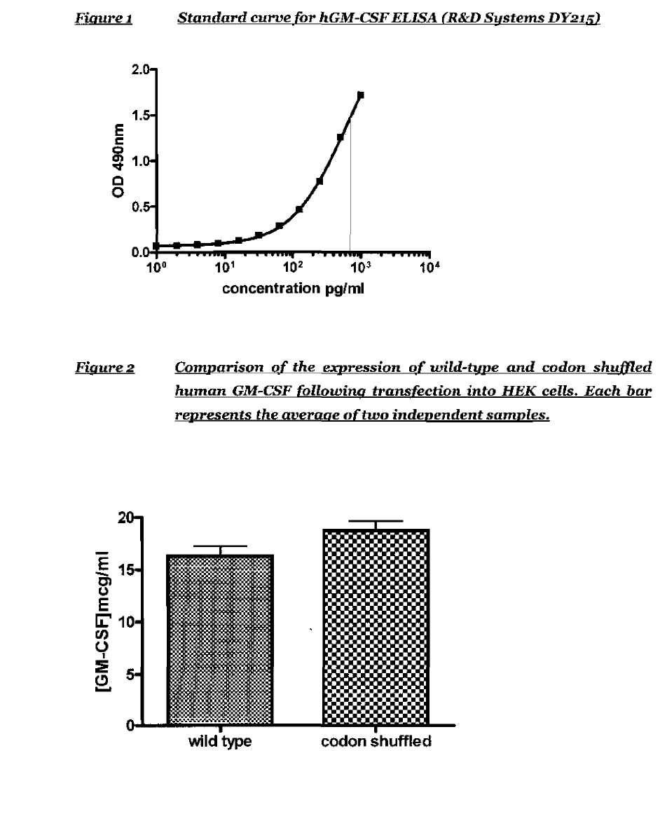

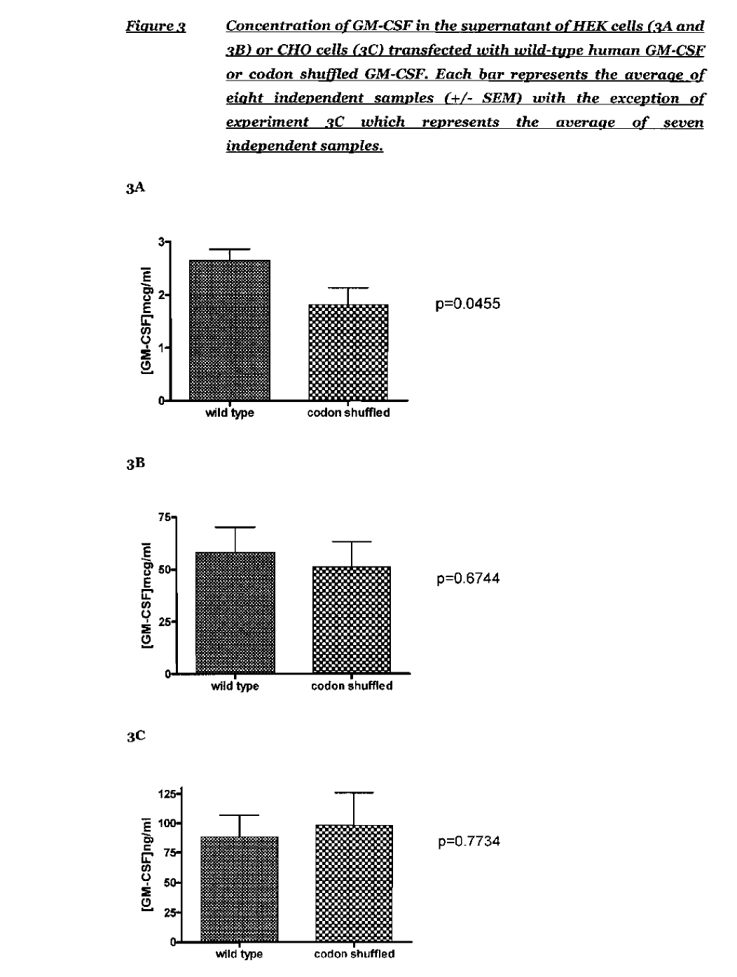

In a preliminary experiment, the concentration of GM-CSF in the supernatants of HEK cells transfected with wild type GM-CSF plasmid and codon shuffled GM-CSF plasmid was assessed. The expression of GM-CSF was assessed by ELISA and converted into concentration using the standard curve shown in FIG. 1. The results presented in FIG. 2 show that the expression from wild-type and codon shuffled GM-CSF plasmids are comparable.

A more detailed investigation of expression levels from wild-type and codon shuffled GM-CSF plasmids was conducted in a series of three independent experiments. In each experiment, two separate batches of plasmid were used for both the wild-type and codon shuffled plasmids. Transfections into HEK cells were performed in quadruplicate (giving a total of eight data points for each plasmid). The mean expression levels of GM-CSF are shown in FIG. 3. Whilst experiment 3A shows that there is a small drop off in expression from the codon shuffled plasmid (p=0.0455), in the repeat experiments (3B and 3C) the expression of wild-type and codon shuffled GM-CSF is comparable (p=0.6744 and 0.7734 respectively), consistent with the preliminary data shown in FIG. 2.

### Summary of Expression Data

The expression data indicates that expression from codon shuffled GM-CSF is comparable to that of wild-type GM-CSF.

### Example 3

### Bioactivity Assay

The human erythroblastoma cell line TF-1 is able proliferate in response to hGM-CSF. Following transient transfection of wild-type and codon shuffled human GM-CSF into HEK293 cells, supernatants were harvested and tested for their activity in a TF-1 bioassay. Dilutions of the cell supernatants were added to TF-1 cells and their proliferation measured after 72 hours.

FIG. 4 and FIG. 5 show comparisons of wild type (wta, wtc) and codon shuffled (csa, csb) GM-CSF plasmids in a TF-1 proliferation assay normalised to concentration of GM-CSF in the supernatant (determined by quantitative ELISA). The results confirm that the GM-CSF expressed from both the wild-type and codon shuffled constructs is bioactive and of equivalent activity.

### Summary of Bioactivity Data

A codon shuffled human GM-CSF gene has been constructed which has 76.9% identity to the wild type DNA sequence but transcribes an amino acid sequence which is identical to the wild type gene product. An expression vector containing the codon shuffled GM-CSF gene (pMNB003) gives comparable levels of expression to a wild type GM-CSF gene inserted into the same expression vector (pMNB004). Furthermore, human GM-CSF expressed from the codon shuffled GM-CSF plasmid and from the wild type GM-CSF plasmid show comparable levels of activity in a cell based bioassay.

### Example 4

### Co-Coating GM-CSF and an Antigen Plasmids

In order to deliver the antigen encoding plasmid and GM-CSF plasmid into the same cell via PMED delivery a co-coating approach was developed. This has the advantage of being to optimise the ratio of antigen and GM-CSF ratio's to yield maximal immunogenicity. This approach is also much easier to standardise for development compared to mixing of beads coated with each plasmid. In addition, it has the advantage of a dual promoter construct with antigen and GM-CSF encoded on same plasmid of being able to develop a generic safety package around GM-CSF which would be of value for all the projects.

It was important to demonstrate that GM-CSF expression did not influence the expression of the antigen and vice versa.

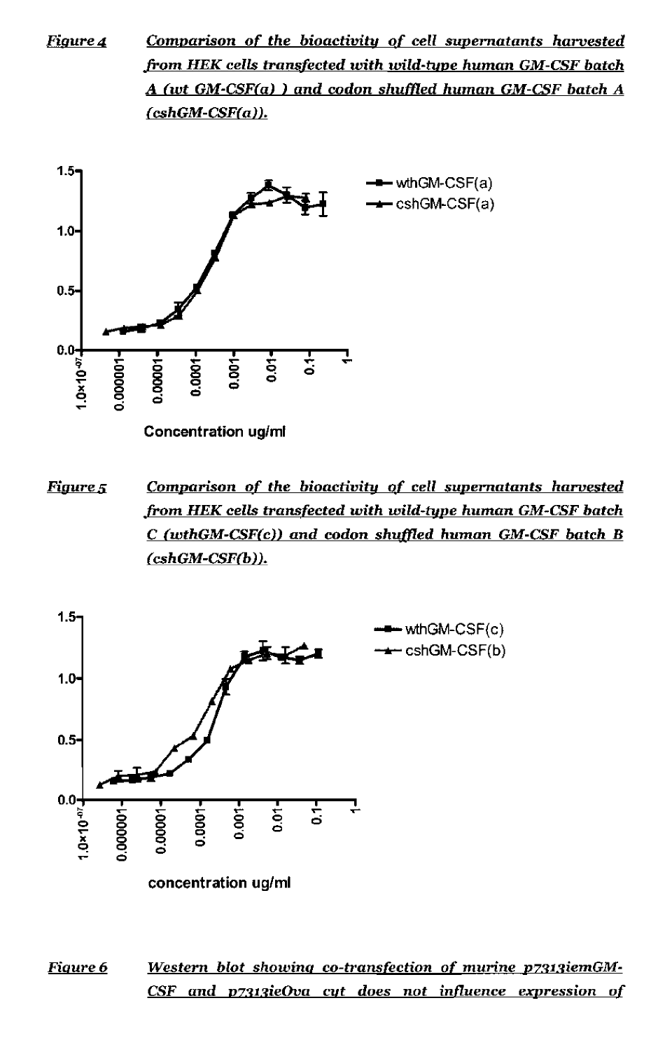

FIG. 6 indicates that co-transfection of p7313ieOva and p7313ie Murine GM-CSF did not alter expression of the antigen (ovalbumin).

1. A non-naturally occurring gene that encodes a protein wherein the non-naturally occurring gene comprises a DNA sequence having less than 80% homology to a cDNA sequence encoding the WT protein which encodes the same protein, characterized in that the codon usage coefficient for the non-naturally occurring gene is not greater than the wild type cDNA sequence.

2. A non-naturally occurring gene as claimed in claim 1, characterized in that the codon usage coefficient for the non-naturally occurring gene is the same as the cDNA sequence Of the wild type gene.

3. The gene of claim 2 characterised in that the level of production of the protein by a host cell which is transfected with said non-naturally occurring gene, in a form which allows expression thereof, is no greater than the level of protein produced by a host cell when it is transfected with the wild type gene.

4. The non-naturally occurring gene according to claim 1 wherein the non-naturally occurring gene encodes a polypeptide which is identical to the mature form of a polypeptide which is encoded by a gene present in the human genome.

5. The non-naturally occurring gene of claim 4 wherein the gene encodes codon-shuffled Granulocyte Macrophage Colony Stimulating factor (GM-CSF).

6. The non-naturally occurring gene of claim 5, wherein the sequence is that of SEQ ID NO. 1.

7. An expression cassette comprising the non-naturally occurring gene of claim 1.

8. A plasmid comprising the expression cassette of claim 7.

9. A vaccine composition comprising (a) the plasmid vector of claim 8, and (b) a polynucleotide encoding a protein against which it is desired to induce an immune response.

10. The vaccine composition of claim 9 wherein the protein against which it is desired to induce an immune response is selected from HCV, HIV, HPV or a tumor associated antigen.

11. A method of inducing an immune response in a mammal, comprising administering to said mammal a vaccine as claimed in claim 8.

12. A method as claimed in claim 10, further comprising the administration of a TLR-agonist.

13. Method of creating a synthetic gene that has less than 80% homology at a DNA level to a cDNA sequence of a wild-type gene wherein the synthetic gene and wild-type cDNA gene encode the same polypeptide comprising:
a) identifying the codons for each amino acid in the wild-type cDNA sequence; and
b) if any amino acid residue is encoded by the wild-type cDNA sequence in at least two locations then moving at least 80% of the codons from its first position where it is found in the wild-type cDNA sequence to a second position not found in the wild-type cDNA sequence. (“said” sequence? Instead of complementary all the time? Save repetition?)

14. A method of producing the synthetic gene of claim 12 wherein at least 90% of the codons have been translocated.

15. Method of creating a synthetic gene that has less than 80% homology at a DNA level to a cDNA sequence of a wild-type gene wherein the synthetic gene and wild-type gene encode the same polypeptide comprising:
a) identifying the codons for each amino acid in the wild-type cDNA sequence; and
b) rearranging the codons so that each codon is found in the synthetic gene in the same proportions as the wild-type cDNA sequence;
wherein the level of protein produced by the cell is not greater than that of the cell when transfected with the wild type cDNA sequence.

16. Method of creating a synthetic gene that has less than 80% homology at a DNA level to a cDNA sequence of a wild-type gene wherein the synthetic gene and wild-type gene encode the same polypeptide comprising:
a) identifying the codons for each amino acid in the wild-type cDNA sequence; and
b) moving a sufficient number of codons from their first position in the wild-type cDNA sequence to a second position not found in the wild-type cDNA sequence;
Wherein the codon usage coefficient of the synthetic genes is not higher than that of the wild-type cDNA sequence.

17. A method of treating a patient comprising the administration of a safe and effective amount of an immunogenic, vaccine or pharmaceutical composition according to claim 8.

18. A method of raising an immune response in a mammal against a disease state, comprising administering to the mammal within an appropriate vector, a nucleotide sequence encoding an antigenic peptide associated with the disease state; additionally administering to the mammal with an appropriate vector, a nucleotide sequence encoding GM-CSF; and further administering to the mammal in imidazoquinoline or derivative to raise the immune response.

19. Use of the plasmid of claim 7 and an imidazoquinoline or derivative thereof in the manufacture of a medicament for enhancing immune responses initiated by an antigenic peptide or protein, the antigenic peptide or protein being expressed as a result of administration to a mammal of a nucleotide sequence encoding for the peptide.


SEQUENCE LISTING (6 sequences)

SEQ ID NO: 1 - DNA, 435 bp, Artificial Sequence
  cshGM-csf coding sequence
atgtggctccagtcgctcctcctgctgggcacggtagcctgctctattagcgcccccgctcggagcccctcaccctccacccagccatgggaacacgtcaacgctatccaggaagcccgcagactgctaaacctgagccgcgacaccgccgccgaaatgaatgagactgtcgaggtgatcagcgagatgtttgacctgcaggaacccacttgcctgcagacacggctcgagctctacaaacagggcctgcgtggcagtctgaccaagttgaagggccctcttaccatgatggcaagccattacaagcagcactgtccgcccacccccgagaccagttgcgcaacccagatcatcacttttgagagctttaaggagaatctgaaggacttcttgctggtgatcccatttgactgctgggaaccggtccaggagtga

SEQ ID NO: 2 - DNA, 458 bp, Artificial Sequence
  Expression cassette
gctagcgcagaggatgtggctccagtcgctcctcctgctgggcacggtagcctgctctattagcgcccccgctcggagcccctcaccctccacccagccatgggaacacgtcaacgctatccaggaagcccgcagactgctaaacctgagccgcgacaccgccgccgaaatgaatgagactgtcgaggtgatcagcgagatgtttgacctgcaggaacccacttgcctgcagacacggctcgagctctacaaacagggcctgcgtggcagtctgaccaagttgaagggccctcttaccatgatggcaagccattacaagcagcactgtccgcccacccccgagaccagttgcgcaacccagatcatcacttttgagagctttaaggagaatctgaaggacttcttgctggtgatcccatttgactgctgggaaccggtccaggagtgagaggcgcgcc

SEQ ID NO: 3 - DNA, 3848 bp, Artificial Sequence
  Full plasmid sequence
ctagcgcagaggatgtggctccagtcgctcctcctgctgggcacggtagcctgctctattagcgcccccgctcggagcccctcaccctccacccagccatgggaacacgtcaacgctatccaggaagcccgcagactgctaaacctgagccgcgacaccgccgccgaaatgaatgagactgtcgaggtgatcagcgagatgtttgacctgcaggaacccacttgcctgcagacacggctcgagctctacaaacagggcctgcgtggcagtctgaccaagttgaagggccctcttaccatgatggcaagccattacaagcagcactgtccgcccacccccgagaccagttgcgcaacccagatcatcacttttgagagctttaaggagaatctgaaggacttcttgctggtgatcccatttgactgctgggaaccggtccaggagtgagaggcgcgccatatgacgtcagatctgtcgacggatccgatctttttccctctgccaaaaattatggggacatcatgaagccccttgagcatctgacttctggctaataaaggaaatttattttcattgcaatagtgtgttggaattttttgtgtctctcactcggaagcaattcgttaaccggaaatacaggaacgcacgctggatggcccttcgctgggatggtgaaaccatgaaaaatggcagcttcagtggattaagtgggggtaatgtggcctgtaccctctggttgcataggtattcatacggttaaaatttatcaggcgcgatcgcggcagtttttcgggtggtttgttgccatttttacctgtctgctgccgtgatcgcgctgaacgcgttttagcggtgcgtacaattaagggattatggtaaatccacttactgtctgccctcctagccatcgagaattctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccggcatgcctgcaggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgcggccg

SEQ ID NO: 4 - DNA, 435 bp, Homo sapien
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagtga

SEQ ID NO: 5 - protein, 144 aa, Homo sapien
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 6 - protein, 127 aa, Homo sapien [sequence omitted]